(19) United States
(12) Patent Application Publication
(10) Pub. No.: US 2024/0415605 A1
(43) Pub. Date: Dec. 19, 2024

(54) ADJUSTABLE HEADREST FOR A DISPLAY UNIT

(71) Applicant: INTUITIVE SURGICAL OPERATIONS, INC., Sunnyvale, CA (US)

(72) Inventors: Charles A. Sweeney, Manteca, CA (US); Ehsan Noohi Bezanjani, Los Gatos, CA (US); Sina Parastegari, Cupertino, CA (US); Allen C. Thompson, Los Altos, CA (US)

(73) Assignee: INTUITIVE SURGICAL OPERATIONS, INC., Sunnyvale, CA (US)

(21) Appl. No.: 18/703,093

(22) PCT Filed: Oct. 20, 2022
(86) PCT No.: PCT/US2022/047301

§ 371 Date: Apr. 19, 2024

Related U.S. Application Data
(60) Provisional application No. 63/270,418, filed on Oct. 21, 2021.

(51) Int. Cl. A61B 90/60; A61B 34/35; A61B 90/00

(52) U.S. Cl. CPC A61B 90/60; A61B 34/35; A61B 2090/372

(57) ABSTRACT

A display unit of a user control system of a telesurgical system includes a support structure, a headrest, and a lens. The headrest is movably connected to the support structure. The lens is below the headrest and includes an optical axis. The headrest is movable to a plurality of positions along a position adjustment axis that is parallel to the optical axis. And, the headrest includes a curved user-facing surface that is shaped to receive a user's head in a way that comfortably aligns the user's eye horizontally and vertically with the optical axis of the lens.

### CLAIM OF PRIORITY

This application claims the benefit of priority to U.S. Patent Application Ser. No. 63/270,418, filed on Oct. 21, 2021, which is incorporated by reference herein in its entirety.

### BACKGROUND

In some applications, a display device can be used in conjunction with other devices and allow a user to observe a scene captured by a camera. For example, in a teleoperated system, a user typically operates a control input device to remotely control (e.g., teleoperate) the motion and/or other functions of a controlled device, such as a manipulator system, at a work site.

An example of such a system is a telesurgical system with which a user operates a control input device to manipulate surgical instruments and other devices to perform a surgical operation at a surgical site. A control input device often includes hand input devices such as pincer grips, joysticks, exo-skeletal gloves, or the like. In some examples of a surgical or other medical task, a hand input device may control a variety of surgical instruments such as tissue graspers, needle drivers, electrosurgical cautery probes, cameras, etc., which perform functions such as holding or driving a needle, grasping a blood vessel, or dissecting, cauterizing, or coagulating tissue.

In various teleoperated systems, a display unit is used in conjunction with the control input device. For example, the display unit can include a display device that displays images depicting a view of a remote work site, or a portion thereof, as captured by a camera at the work site.

However, teleoperated systems may provide a viewing device that has a restrictive viewing area (e.g., the user must peer through viewports or eyepieces) and is static and rigid in its position relative to the user. In such cases, the user may be required to conform their head and body position to the viewing device to effectively operate the teleoperated system. In addition, viewer design may require the use of an ocular lens or eyepiece lens assembly (i.e., closest to the eye) that has a relatively small associated volume in which the user's pupil should be positioned in order to effectively view the image transmitted through the ocular lens or eyepiece assembly. This volume may be defined by horizontal and vertical distances from the lens's optical axis, in addition to a distance from the lens along the lens's optical axis. There is a need to quickly, effectively, and comfortably guide the user's head into a position with reference to the viewing device so that the pupil is positioned within this volume, and then effectively and comfortably maintain the user's head position in position to maintain the pupil within this volume.

### SUMMARY

The user control system of telesurgical systems commonly includes a component or portion of the system by which the user views a remote site of interest, e.g., a surgical scene. Such display units may be configured to allow the user to rest their head in, on, or near the device while teleoperating a manipulator system to conduct a surgical procedure. A challenge with such user control systems and display units of such systems is actively positioning or guiding a user to position their head and eyes relative to the display unit for proper viewing through one or more viewports. Each viewport includes an ocular lens or eyepiece assembly for which there is an optimal eye position.

In the case of dual viewports, one for each eye of the user (typically used for stereoscopic displays), a user sitting (or standing or kneeling) at the user control system/console looks through two lenses mounted in the display unit to view the surgical site. In such cases, the display unit can include a mechanism that allows the user to adjust the spacing between the lenses to match the user's interpupillary distance. However, the horizontal position (left-right, side-to-side) of the user's eyes, the vertical position (up-down) of the eyes, and the distance of the eyes away from the lenses (in-out; this distance is sometimes referred to as “relief” and generally refers to a distance along an axis parallel to the optical axis of the lens/viewport) still need to be positioned properly for proper viewing into the display unit.

The inventor(s) have devised, among other things, an adjustable headrest that is configured to guide users with various head sizes and shapes to place their pupils within a relatively small optimal volume in space relative to lenses of a display unit of a user control system. The devices and methods of the present disclosure are configured to guide the user to position their eyes/pupils in three dimensions, e.g., left-right relative to the lenses (referred to herein arbitrarily as X-axis), vertically relative to the lenses (referred to herein arbitrarily as Y-axis), and nearer or farther away from a major face of the lenses (referred to herein arbitrarily as Z-axis). Further, the devices and methods of the present disclosure are intended to encompass embodiments in which a user's single eye is positioned with reference to a single ocular lens or eyepiece assembly, both user's eyes are positioned with reference to separate and individual ocular lenses or eyepiece assemblies each corresponding to an individual one of the user's eyes (e.g., for stereoscopic viewing), or both. It should be understood that aspects of this disclosure described in terms of both user's eyes encompass similar aspects for a user's single eye, and vice-versa.

Display units in accordance with this disclosure include a contoured, adjustable headrest that guides and positions eyes of a user relative to lenses of the display unit. The headrest includes a contoured shape generally in the X-Z plane, which is designed to guide the eyes of the user via forehead placement on the headrest along the X-axis. The headrest also includes an angled, contoured shape generally in the Y-Z plane to accommodate a variety of forehead shapes and to guide positioning of the eyes along the Y-axis. The headrest is also offset from the optical axis of the lenses by a fixed predetermined distance along the Y-axis to further guide the position of the eyes along the Y-axis, which can be selected based on empirical evidence regarding the distance between a range of user forehead dimensions and eyes/pupils dimensions. Finally, the headrest is movable along the Z-axis to actively position the eyes of the user an optimal distance (relief) from the face of the lenses. Devices and methods in accordance with this disclosure can be used independent of or in conjunction with systems that adjust the distance between the lenses of the display unit to accommodate the user's interpupillary distance.

Examples according to this disclosure include a display unit of a user control system of a telesurgical system. The display unit includes a support structure, a headrest, and a lens. The headrest is movably connected to the support structure. The lens is below the headrest and includes an optical axis. The headrest is movable to a plurality of positions along a position adjustment axis that is parallel to the optical axis. And, the headrest includes a curved user-facing surface that is angled toward the lens from an upper portion to a lower portion of the curved user-facing surface.

Each of the non-limiting examples of the present patent application can stand on its own or can be combined in various permutations or combinations with one or more of the other examples.

This Summary is intended to provide an overview of subject matter of the present patent application. It is not intended to provide an exclusive or exhaustive explanation of the invention. The detailed description is included to provide further information about various aspects of the inventive subject matter of the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a diagrammatic view depicting an example telesurgical system.

FIG. 2 is a front elevation view depicting a user control system including an example display unit in accordance with examples of this disclosure.

FIGS. 3A and 3B are side elevation side and top plan views depicting another example display unit in accordance with examples of this disclosure.

FIGS. 4A and 4B are perspective and section views depicting another example display unit in accordance with examples of this disclosure.

FIGS. 5A-5C depict an example contoured headrest that is configured to be movably coupled to a display unit of a user control system of a telesurgical system.

FIGS. 6A-6C depict an example cushion for a contoured headrest, which includes a three-dimensional lattice structure.

### DETAILED DESCRIPTION

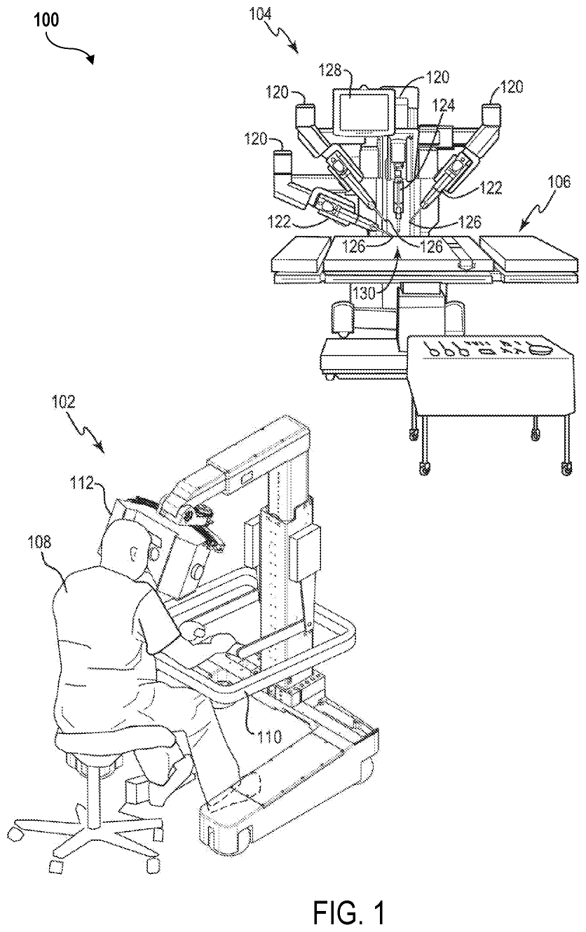

FIG. 1 is a diagrammatic view depicting example telesurgical system 100, which includes user control system 102 and manipulator system 104.

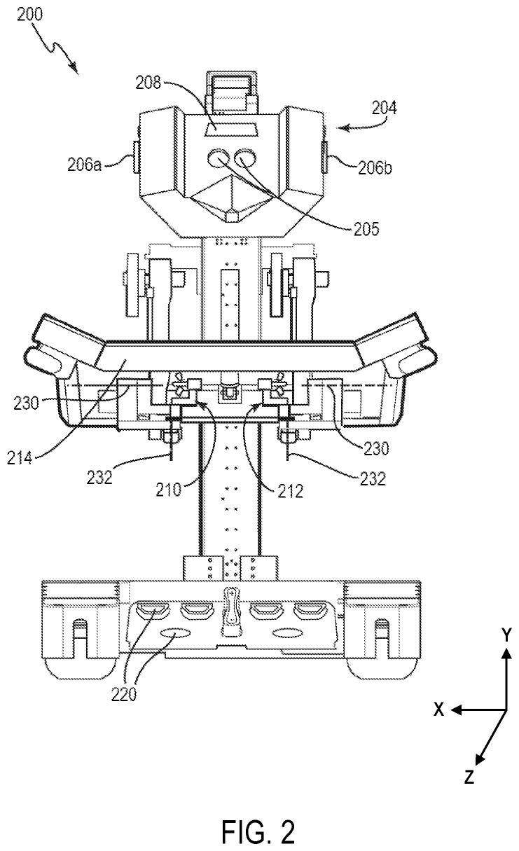

In this example, user control system 102 includes one or more control input devices which are contacted and manipulated by the user's hands, e.g., one control input device for each hand (e.g., 210,212 shown in FIG. 2), optional ergonomic support 110, and display unit 112. The control input devices are supported by user control system 102 and can be mechanically grounded or ungrounded. Ergonomic support 110 (e.g., forearm rest) can be provided in some implementations, on which user 108 can rest his or her forearms while grasping control input devices. For example, the control input devices can be positioned in a workspace inward (away from user 108) beyond support 110. In some examples, user 108 performs surgical tasks at a work site near manipulator system 104 during a surgical procedure by controlling manipulator system 104 using the control input devices.

Display unit 112 displays images for viewing by the user 108. For example, the images can be displayed by a display device in the display unit, such as one or more display screens, projectors, or other devices. Display unit 112 as a whole can be moved in various degrees of freedom to accommodate the user's viewing position and/or to provide control functions. Additionally, display unit 112 includes a contoured movable headrest that is configured to guide user 108 to position eyes of user 108 relative to one or more lenses/viewports of the display unit, as described in greater detail below.

In the example of teleoperated system 100, displayed images can depict a work site at which the user is performing various tasks via control of the control input devices. In some examples, the images displayed by the display unit 112 can be received by the user control system 102 from one or more image capture devices arranged at a remote work site. In other examples, the images displayed by the display unit can be generated by the display unit (or by a connected other device or system). In an example of a surgical procedure using teleoperated system 100, display unit 112 can display images of a physical surgical site at a patient near manipulator system 104, or a generated virtual representation of a surgical site or a combination of physical and virtual sites (e.g., augmented reality), and can display real or virtual instruments of manipulator system 104 controlled by the control input devices of user control system 102.

Display unit 112 can provide a two-dimensional image and/or a stereoscopic image of, for example, an end effector of manipulator instrument 126 and the surgical site. A stereoscopic image provides three-dimensional depth cues to permit user 108 to assess relative depths of instruments and patient anatomy and to use visual feedback to steer manipulator instruments 126 using control input devices to precisely target and control instrument features.

When using user control system 102, user 108 can sit, stand, or kneel in front of user control system 102, position or have positioned his or her eyes in front of display unit 112 (and/or move the display unit 112 to a position/orientation of his or her eyes), grasp and manipulate the control input devices, e.g., one in each hand, and rest his or her forearms on ergonomic support 110 as desired. In examples of this disclosure, it is assumed that user 108 is in a generally upright seated, standing, or kneeling position relative to and forward facing (eyes facing) user control systems and display units thereof. Spatial frames of reference described with reference to examples of this disclosure therefore generally include three dimensions corresponding to left-right/side-to-side (X-axis), vertical or up/down (Y-axis), and near to, far from (Z-axis) the user control system and display unit.

Additionally, as noted above, display unit 112 and other display units in accordance with this disclosure are capable of adjustment in two different frames of reference. Display unit 112 as a whole can be moved in various degrees of freedom relative to user control system 102, which in this control system reference frame is considered ground. Additionally, display unit 112 includes a contoured movable headrest that is configured to guide user 108 to position and actively position user 108's eyes relative to the display unit, which in this display unit reference frame is considered ground.

Referring again to FIG. 1, teleoperated system 100 also includes manipulator system 104, which can be teleoperated via user control system 102. In this example, manipulator system 104 is positioned near operating table 106 (e.g., table, bed, or other support) on which a patient may be positioned. Optionally, some or all components of manipulator system 104 are mounted to table 106. Work site 130 can be provided on operating table 106, e.g., on or in a patient, simulated patient or model, etc. (not shown). In other implementations, a work site can be a different site or area at which tasks are to be performed using a manipulator system. Teleoperated manipulator system 104 includes a plurality of manipulator arms 120, each coupled to instrument assembly 122. Instrument assembly 122 includes, for example, instrument 126. In some examples, instruments 126 may include surgical instruments, and such instruments can include a surgical end effector at its distal end, e.g., for treating tissue of a patient.

One or more of instruments 126 can include an image capture device (e.g., a camera), such as one or more cameras included in endoscope assembly 124, which can provide captured images of a portion of the work site. Captured images can be transmitted to display unit 112 of user control system 102 for output. Display device 128 can be included on manipulator system 104 to display captured images and/or other information related to a procedure being performed at the work site. The camera or other image capture device in such examples can be moved in multiple degrees of freedom, e.g., based on translation and rotation of portions of manipulator arm 120 holding the camera.

In an example of a surgical procedure using teleoperated system 100, manipulator system 104 can be positioned close to a patient for surgery (or a simulated patient for training), where it can remain stationary until a particular surgical procedure or stage of a procedure is completed. Additionally, user control system 102 can be positioned in various locations relative to manipulator system 104, e.g., in a sterile surgical field close to manipulator system 104 and work site 130, in the same room as manipulator system 104 and work site 130, or remotely from manipulator system 104 and work site 130, e.g., in a different room, building, or other geographic location. The number of teleoperated instruments 126 used at one time, and/or the number of arms 120 used in manipulator system 104, may depend on the procedure to be performed and the space constraints within the available area, among other factors.

Manipulator arms 120 and/or instrument assemblies 122 can be controlled to move and articulate instruments 126 in response to manipulation of control input devices by user 108, so that the user can perform tasks at work site 130. For example, the user can direct surgical procedures at internal surgical sites through minimally invasive surgical apertures. One or more actuators coupled to manipulator arms 120 and/or instrument assemblies 122 may output force to cause links or other portions of arms 120 and/or instruments 126 to move in particular degrees of freedom in response to control signals received from the control input devices.

Some implementations of teleoperated system 100 can provide different modes of operation. For example, in a non-controlling mode (e.g., safe mode) of teleoperated system 100, the controlled motion of manipulator system 104 is controllably decoupled (disconnected) from the control input devices such that movement and other manipulation of the control input devices do not cause motion of manipulator system 104. In a controlling mode of teleoperated system 100 (e.g., following mode), however, motion of manipulator system 104 can be controllably coupled (connected) to the control input devices such that movement and other manipulation of the control input devices causes motion of manipulator system 104, e.g., during a surgical procedure. Such modes of operation can be implemented for manipulator system 104 as a whole or for portions thereof, e.g. for each of manipulator arms 120 and associated instrument assemblies 122.

Teleoperated system 100 may also include a control system in/with user control system 102 and/or external to/separate from user control system 102 (e.g., in communication with the user control system). As user 108 moves control input device(s) of user control system 102, sensed spatial information and sensed orientation information is provided to the control system based on the movement of the control input devices. Other user input is also provided to the control system, e.g., user input received at display unit 112, and/or activation of other input devices. The control system can provide control signals to manipulator system 104 to control the movement of arms 120, instrument assemblies 122, and instruments 126 based on the received information and user input.

Some implementations according to this disclosure can include one or more components of a teleoperated medical system such as a da Vinci® surgical system (e.g., a Model IS3000 or IS4000, marketed as the da Vinci Si® or da Vinci Xi® surgical system), commercialized by Intuitive Surgical, Inc. of Sunnyvale, California. Features disclosed herein may be implemented in various ways, including in implementations at least partially computer-controlled, controlled via electronic control signals, manually controlled via direct physical manipulation, etc. Implementations on da Vinci® surgical systems are merely examples and are not to be considered as limiting the scope of the features disclosed herein. For example, different types of teleoperated systems having manipulator systems at work sites can make use of features described herein. Other, non-teleoperated systems can also use one or more described features, e.g., various types of control systems and devices, peripherals, etc.

FIG. 2 is a front elevational view of example user control system 200 including control input devices and a display system. User control system 200 including display unit 204 can be similar to user control system 102 and display unit 112 of FIG. 1.

User control system 200 includes display unit 204 which can display images during a procedure implemented by a teleoperated system. The images can be captured by an image capture device and depict a physical work site at which a task is performed, such as a surgical site displayed during a surgical procedure, or can depict a generated representation of a virtual work site. Display unit 204 can also display other information, such as a graphical user interface allowing selection of commands and functions, status information, alerts and warnings, notifications, etc. Such information can be displayed in combination with (e.g., overlaid on) a view of a work site, or without a work site view.

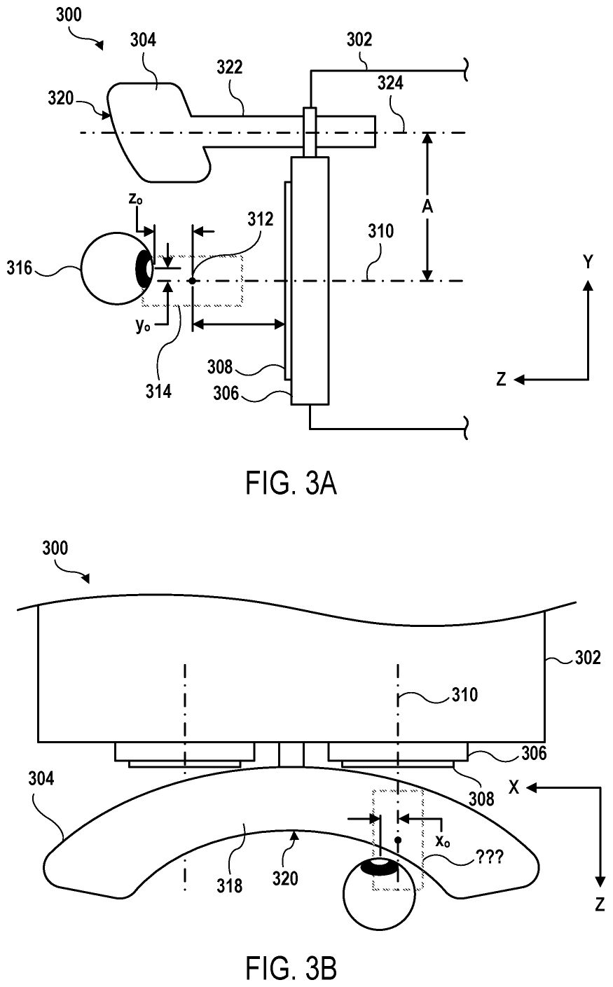

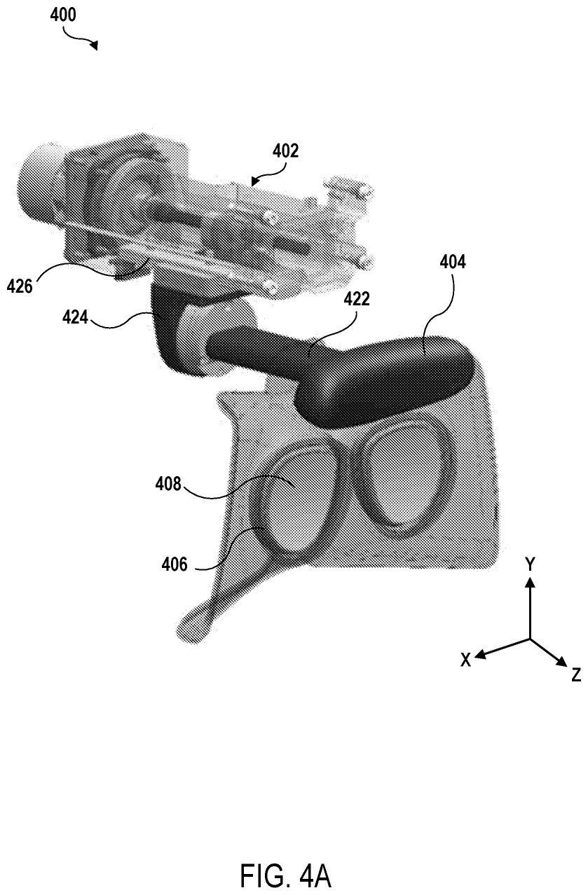

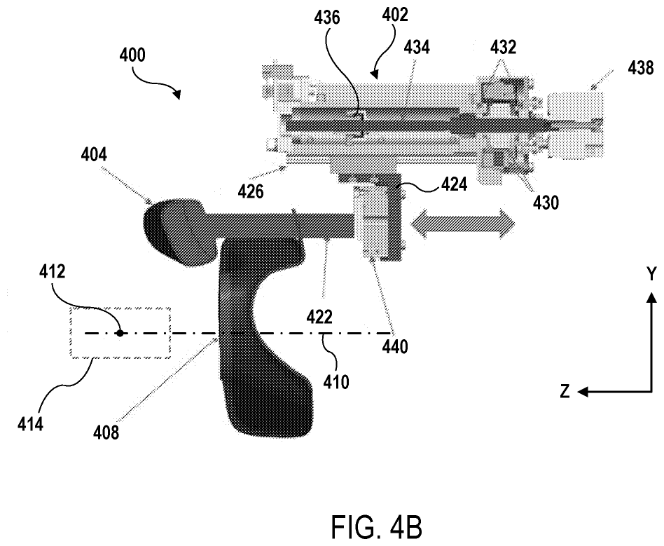

Display unit 204 includes two viewports 205, hand input devices 206a, 206b, and adjustable headrest 208, which is schematically depicted in the example of FIG. 2. Headrest 208 can guide a user to position and actively position the user's head such that the user's eyes are aligned with and at an appropriate position relative to viewports 205 to view images displayed by display unit 204. In some examples, one or more display screens can be provided behind viewports 205 which display images to the viewing user. In some implementations, one or more display screens or other display devices can be used instead of viewports 205. Additionally, each of viewports 205 may include a lens that defines an optical axis, as will be described in greater detail with reference to FIGS. 3A-4B.

Example display unit 204 and adjustable headrest 208 are configured to guide a user to position and/or actively position eyes/pupils of a user in three dimensions/degrees of freedom of movement. For example, headrest 208 can include a contoured shape in the X-Z plane, which is designed to guide the eyes of the user along the X-axis when the forehead of the user is received by/engages the contoured shape of the headrest. Headrest 208 can also include an angled, contoured shape in the Y-Z plane to accommodate a variety of forehead shapes and to guide placement of the eyes along the Y-axis. Headrest 208 can also be offset from the optical axis of the lenses of viewports 205 by a fixed predetermined distance along the Y-axis to further guide the position of the eyes along the Y-axis. Moreover, headrest 208 can be movable along the Z-axis to actively position the eyes of the user an optimal distance, or relief from the face of lenses of viewports 205, e.g. an optimal distance along or parallel to the optical axis of the lenses.

User control system 200 can include one or more optional input control devices to allow a user to adjust or otherwise manipulate the position and/or orientation of display unit 204 as a whole relative to user control system 200 and/or portions thereof (e.g., with respect to control input devices 210 and 212, described below). For example, display unit 204 includes hand input device 206a positioned on the left side of the display unit and hand input device 206b on the right side of display unit 204. Hand input devices 206a and 206b can receive user input to cause display unit 204 to change its orientation and/or position, e.g., to provide ergonomic adjustments for more user comfort. Such one or more hand input devices can alternatively or additionally be positioned at other areas or components of user control system 200.

User control system 200 also includes control input devices 210 and 212 for user manipulation to control motion of one or more components of manipulator system 104. For example, a user can rest their forearms on ergonomic support 214 while gripping portions of control input devices 210 and 212, with one control input device in each hand. Ergonomic support 214 can also be adjustable in height for different users. The control input devices may include one or more of any number of a variety of input devices manipulable by the user, such as kinematically linked (mechanically grounded) hand grips, joysticks, trackballs, data gloves, trigger-guns, hand-operated controllers, voice recognition devices, touch screens, and the like.

Each control input device 210 and 212 can include grip portions that are moveable in a plurality of degrees of freedom. For example, each control input device 210 and 212 can control motion and functions of an associated arm assembly of a manipulator system, for example, arm assembly 120 of manipulator system 104 in FIG. 1. Control input device 210 can be moved in a plurality of degrees of freedom to move and operate one corresponding end effector of the manipulator system in corresponding degrees of freedom, and control input device 212 can be moved in a plurality of degrees of freedom to move and operate a different corresponding end effector of the manipulator system in corresponding degrees of freedom.

In some cases, control input devices 210 and 212 are provided with the same degrees of freedom as associated instruments of the manipulator system to provide the operator with telepresence, e.g., the perception that the control input devices are integral with the instruments so that the operator has a strong sense of directly controlling instruments as if present at the work site. In other implementations, control input devices 210 and 212 may have more or fewer degrees of freedom than the associated instruments. Additionally, the control input devices can be manual input devices which move in all six Cartesian degrees of freedom, and which may also include an actuatable grip portion (e.g., handle) for actuating manipulator instruments, e.g., for closing grasping jaws, applying an electrical potential to an electrode, delivering a medicinal treatment, and the like. A grip function, such as moving two grip portions of a control input device together and apart in a pincher movement, can provide an additional mechanical degree of freedom (i.e., a grip DOF).

Example user control system 200 also includes foot controls 220 positioned below control input devices 210 and 212. Foot controls 220 can be depressed, slid, and/or otherwise manipulated by a user's feet to input various commands to a control system of a teleoperated system while the user is operating user control system 200. Foot controls 220 or other controls can be considered “control input devices” that can control one or more functions or operations of the manipulator system being teleoperated via user control system 200.

In some implementations, one or more user presence sensors can be positioned at one or more locations of user control system 200 to detect the presence of a user operating and/or located next to or near to the user control system. For example, user presence sensors can be positioned on display unit 204 and sense a presence of a user's head aligned with viewports 205. For example, an optical sensor can be used for a presence sensor, where the optical sensor includes an emitter and a detector and an interruption of an optical beam is sensed by the detector when the user's head is positioned to view the output of display unit 204 and the user is in a proper position to use control input devices 210 and 212. In some implementations, hand input devices 206 and/or adjustable headrest 208 can be used to sense user presence.

FIG. 3A is a side elevation view, and FIG. 3B is a corresponding top plan view, depicting example display unit 300 in accordance with examples of this disclosure. Display unit 300 can be included in/coupled to a user control system of a telesurgical system. In FIGS. 3A and 3B, display unit 300 includes support structure 302, contoured movable headrest 304, viewports 306, and lenses 308. Headrest 304 is movable along the Z-axis closer to and farther away from the support structure 302 of display unit 300, viewports 306, and lenses 308. Two viewports 306, one for each eye of a user, each include a lens 308. And, each lens 308 defines optical axis 310 and a point 312 on optical axis 310, which will be referred to as focal point 312.

Images or other information displayed inside display unit 300 through viewports 306 and lenses 308 may be optimally viewed by users by positioning the pupils of the users at focal point 312. However, it may be impractical for users to position themselves or for mechanisms of display unit 300, e.g. headrest 304 to guide the users to position or to actively position the users with their pupils at the focal points 312 of lenses 308. As such, a focal zone 314 can be defined, which represents a volume of space within which the eyes of users of display unit 300 can be positioned for acceptable viewing of information displayed by the display unit through lenses 308 of viewports 306.

An eye 316 of a user is depicted in FIGS. 3A and 3B with the pupil of eye 316 within focal zone 314 but not precisely at focal point 312. At the position of eye 316 schematically depicted in FIGS. 3A and 3B, the user is in an acceptable position for viewing information displayed by display unit 300 and the pupil of eye 316 is offset from focal point 312 in three directions indicated by xₒ, yₒ, zₒ, which represent the offset distance of the pupil in the X, Y, and Z dimensions from focal point 312. Contoured movable headrest 304 of display unit 300 (and other example adjustable headrests and display units in accordance with this disclosure) is configured to both passively guide the user to position eye 316 within focal zone 314, and to actively position the user such that eye 316 is within focal zone 314.

Aspects associated with headrest 304 that guide eye 316 to the correct position with reference to lens 308 along one of the individual X, Y, or Z axes may be implemented as separate embodiments. Or, they may be combined in other embodiments to guide eye 316 to the correct position with reference to lens 308 along two or all three of the individual X, Y, and Z axes in various combinations. Persons of skill in the art will understand that aspects described in combination with one another may optionally be implemented individually with reference to a single one of the X, Y, and Z axes. In the example of FIGS. 3A and 3B, headrest 304 is configured to guide the user to position and/or actively position eye 316 in three dimensions/degrees of freedom of movement. For example, headrest 304 can include a contoured shape in the X-Z plane, which is designed to guide the eyes of the user along the X-axis when the forehead of the user is received by/engages the contoured shape of the headrest. Referring to FIG. 3B, headrest 304 includes a toroidal section defining concave user-facing portion 318 in which is defined a forehead contact surface that is generally shaped as a section of a toroid taken close to the hole in the toroid. Concave user-facing portion 318 of headrest 304 includes curved user-facing surface 320. User-facing surface 320 is curved in two planes. Referring to FIG. 3B, user-facing surface 320 is a concave curved surface in the X-Z plane, and referring to FIG. 3A, user-facing surface 320 is a convex curved surface in the Y-Z plane. Additionally, as shown in FIG. 3A, user-facing surface 320 is angled, from an upper to a lower portion of the curved user-facing surface toward support structure 302, viewing portal 306, and lens 308. Stated another way, user-facing surface 320 is both convex in the Y-Z plan as shown, and as a whole the convex curve slopes from top to bottom away from the user and toward support structure 302, viewing portal 306, and lens 308.

The concave curvature of user-facing surface 320 in the X-Z plane is configured to guide eye 316 of the user along the X-axis when the forehead of the user is received by/engages the contoured shape of the headrest. For example, the concave curvature of user-facing surface 320 is selected to comfortably match/mate with a plurality of users with differently shaped and sized foreheads such that the central region of the forehead settles around an axis bisecting headrest 304 in the X-Z plane, thereby generally centering the head of the user and also the eyes of the user along the X-axis. Empirical measurement may be used to determine a wide range of user forehead dimensions to be accommodated. In addition, or alternatively, available anatomical statistics may be used to determine a range of user forehead dimensions to be accommodated. For example, the 10-90th percentile or the 5-95th percentile of forehead widths may be accommodated. In the stereoscopic implementation shown in FIG. 3B, the axis bisecting headrest 304 is aligned with the vertical Y-Z plane separating the two viewing portals 306 and lenses 308. The lenses can be adjusted to accommodate the user's interpupillary distance. In implementations for a single viewing portal 306 and lens 308, the axis bisecting headrest 304 will be horizontally offset in the X direction away from optical axis 310.

The slope/angle and convex curvature of user-facing surface 320 in the Y-Z plane is configured to accommodate a variety of forehead shapes and to guide placement of eye 316 along the Y-axis. Again, empirical measurement or available anatomical statistics may be used to determine a wide range of user forehead dimensions to be accommodated in, for example, the 10-90th percentile or the 5-95th percentile of forehead slopes. User forehead profile shapes range from very prominent (close to vertical) to sloped (sloping from bottom to top away from the viewer), and the sloped/angled configuration of user-facing surface 320, which from top to bottom slopes/angles away from the forehead of the user and toward support structure 302, viewer portals 306, and lenses 308 of display unit 300, is configured to accommodate these many user forehead shapes.

Additionally, increased surface area contact between skin of the user and headrest 304 can lead to poor heat transfer or concentration of heat in the contact area, which can over longer periods of time can lead to user discomfort. The angled and convex curved configuration of user-facing surface 320 is therefore also configured to generally reduce the surface area contact between headrest 304 and the forehead of the user, which can assist with user comfort over longer periods of time by reducing such heat concentration and resultant perspiration.

Thus, in some implementations user-facing surface 320 is compound-curved surface (curved in two planes, the Y-Z plane and the X-Z plane in the examples of FIGS. 3A and 3B), which is configured to guide users to position their eyes in a relatively small optimal/acceptable focal zone 314 in two-dimensions along the X and Y-axes (or in other words, horizontally (right-left; side-to-side) and vertically (up-down)).

Headrest 304 is also offset from optical axis 310 of lens 308 by a fixed predetermined distance A along the Y-axis. Referring to FIG. 3A, headrest 304 is coupled to or integral with shaft 322, which can be configured to be coupled to a mechanism that moves headrest 304 along the Z-axis. Headrest 304 can be movably coupled to support structure 302 of display unit 300 a fixed distance A between a central longitudinal axis 324 of shaft 322 and optical axis 310 to guide the eyes of a variety of users with a variety of head shapes and eye positions along the Y-axis close to or within focal zone 314.

And, headrest 304 can be actively moved (moved with a motor) along the Z-axis to actively position eye 316 along the Z-axis close to or within viewing zone 316. Headrest 304 is coupled to or integral with shaft 322 so that the headrest and shaft move along the Z-axis as a single piece. Shaft 322 is coupled to a mechanism/actuator of display unit that moves headrest 304 along the Z-axis, as described in more detail below with reference to the examples of FIGS. 4A and 4B. The mechanism/actuator that translates headrest 304 along the Z-axis can be, for example, controlled by one or more user input devices of a user control system with which display unit 300 is employed.

The design of display unit 300 and contoured movable headrest 304 (and other example display units and headrests in accordance with this disclosure) may more efficiently and satisfactorily position the eyes of a variety of users than, for example, a system including a separate mechanically grounded headrest that each user would have to adjust in Y and Z for comfort, and an adjustable viewer assembly that is also moved in Y and Z to align the optical axis with the pupil once the separate headrest is adjusted for user comfort. In example display units according to this disclosure, therefore, once the viewer Y-axis predetermined offset A is selected and the Z-axis headrest adjustment is made, the user can easily remove the head from the viewer and then replace the head to replace the head against the headrest to resume a good pupil placement in X, Y, and Z for viewing.

FIGS. 4A and 4B depict portions of another example display unit 400 including contoured movable headrest 404 in accordance with this disclosure. Referring to FIGS. 4A and 4B, display unit 400 includes linear actuator 402, headrest 404, viewports 406, and lenses 408, which define optical axis 410, focal point 412, and focal zone 414. Headrest 404 is coupled to or integral with shaft 422 so that the headrest and shaft move along the Z-axis as a single piece. Shaft 422 is coupled to sled 424, which is slidably coupled to rail 426. Linear actuator 402 includes motor 430, bearings 432, ball screw 434, ball screw nut 436, and encoder 438. Sled 424 is also coupled to and moves with ball screw nut 436.

In a similar manner as described with reference to the example of FIGS. 3A and 3B, display unit 400 and contoured adjustable headrest 404 are configured to guide a user to position and/or actively position eyes/pupils of a user in three dimensions/degrees of freedom of movement. For example, headrest 404 includes a concave curved shape in the X-Z plane, which is designed to guide the eyes of the user along the X-axis when the forehead of the user is received by/engages the contoured shape of the headrest. Headrest 404 also includes an angled, convex curved shape in the Y-Z plane to accommodate a variety of forehead shapes and to guide placement of the eyes along the Y-axis. Headrest 404 can also be offset from optical axis 410 of lenses 408 by a fixed predetermined distance along the Y-axis to further guide the position of the eyes along the Y-axis.

Linear actuator 402 of display unit 400 is configured to move/adjust the position of head rest 404 along the Z-axis to actively position the eyes of the user an optimal distance (relief) from the face of lenses of viewports 406, e.g. an optimal distance along or parallel to optical axis 410 of lenses 408. In operation, linear actuator 402 can be controlled by a user input control/device to move headrest 404. For example, display unit 400 can be included in/coupled to a user control system of a telesurgical system. The user control system can include one or more user input devices, including, for example, user input devices 206a and 206b of user control system 200 of FIGS. 1 and 2. The user can manipulate the one or more user input devices, which directly or via a control system communicates with encoder 438 to cause motor 430 to rotate ball screw 434. As ball screw 434 rotates, ball screw nut 436 moves along the Z-axis. Ball screw nut 436 is coupled to sled 424, which is coupled to shaft 422 of headrest 404 and slidably connected to rail 426. Thus, as ball screw nut 436 is moved by rotation of ball screw 434, headrest 404 moves along the Z-axis.

Linear actuator 402 can be configured to move headrest 404 between a predetermined minimum extension distance and a predetermined maximum extension distance, which range of positions of headrest 404 can be based on the optics of lenses 408, i.e., based on focal point 412 and focal zone 414. The optimal (focal point) or at least acceptable (focal zone) viewing position of display unit 400 is based at least in part on the optics of lenses 408. And, placing a user with their eyes in, for example, focal zone 414 is also based on the size and shape of the head of the user and the position of the eyes of the user in/on their head. As such, linear actuator 402 can be configured to move headrest 404 between a predetermined minimum extension distance and a predetermined maximum extension distance that are selected to guide a plurality of users with different head sizes, shapes, and anatomical configurations to position their eyes within focal zone 414. In one example, the difference between the predetermined minimum extension distance and the predetermined maximum extension distance, i.e., the distance that accommodates users with prominent foreheads and users with sloping foreheads is approximately 60 millimeters (2.36 inches).

To improve user experience and provide increased control over movement of headrest 404, display unit 400 includes sensor 440 positioned between sled 424 and shaft 422. Sensor 440 can be, for example, a force sensor that is communicatively connected to a control system communicating with encoder 438 and controlling actuation of motor 430. A controller controlling motor 430 can be configured to receive one or more signals from sensor 440, which signals are indicative of a load from a user on headrest 404 along the Z-axis. The controller can then control linear actuator 402 (e.g., motor 430) to move headrest 404 with a force magnitude that is a function of the load of the head of the user on headrest 404. This force-controlled movement of headrest 404 can be configured to improve user comfort and to reduce or eliminate the chance that linear actuator 402 applies excessive forces on the forehead of the user. In an example, the control system can be configured to cause linear actuator 402 to apply a relatively small bias force on headrest 404 and the user can exert a relatively small force against headrest 404 to push it closer to display unit 400. Additionally, user input devices can be provided that allow the user to adjust the position of headrest 404 such as to retract the headrest further away from display unit 400.

Example display unit 400 of FIGS. 4A and 4B includes a ball screw linear actuator 402. However, other mechanisms can be employed to adjust/move a headrest of a display unit in accordance with this disclosure. For example, other mechanical, electromechanical, hydraulic, pneumatic, or piezoelectric actuators can be employed to move an adjustable headrest of a display unit in accordance with this disclosure. As examples, a geared linear actuator or a kinematic mechanism/linkage could be employed to move headrest 404.

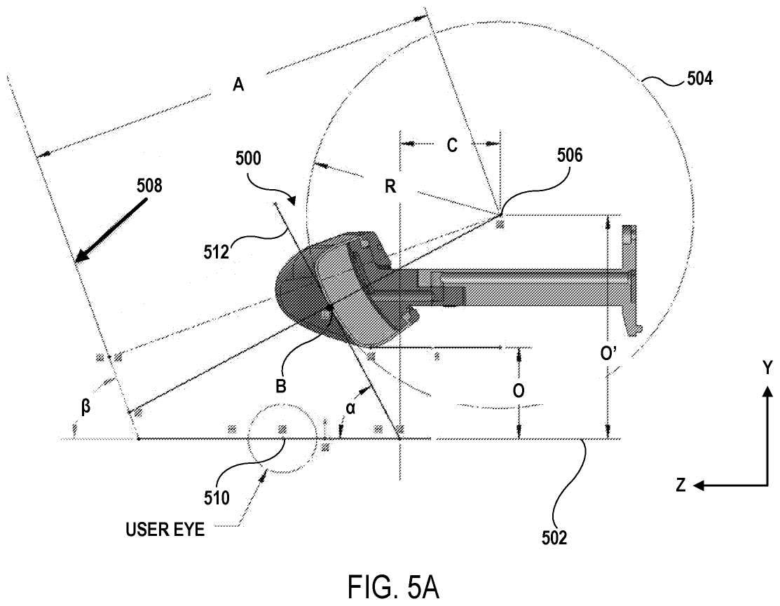

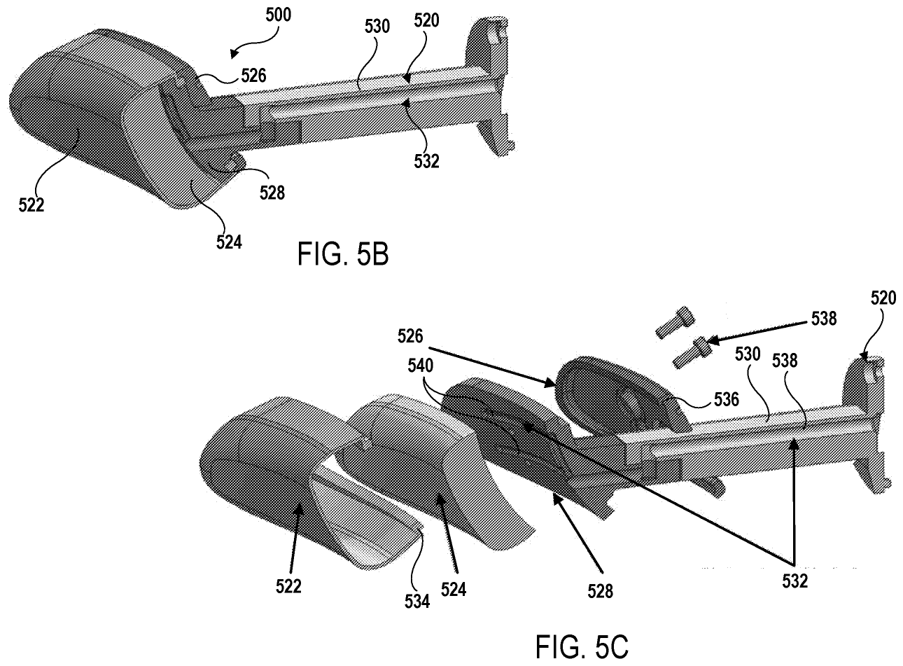

FIGS. 5A-5C depict an example contoured headrest 500 that is configured to be movably coupled to a display unit of a user control system of a telesurgical system. FIGS. 5A-5C depict a vertical cross-section at the centerline of headrest 500, parallel to and midway between two optical axes (one each for the left and right eyes) of a display unit to which the headrest can be attached.

In a similar manner as described with reference to the example of FIGS. 3A-4B, contoured headrest 500 is configured to guide a user to position and/or actively position eyes/pupils of a user in three dimensions/degrees of freedom of movement. For example, headrest 500 includes a concave curved shape in the X-Z plane, which is designed to guide the eyes of the user along the X-axis when the forehead of the user is received by/engages the contoured shape of the headrest. Headrest 500 also includes an angled, convex curved shape in the Y-Z plane to accommodate a variety of forehead shapes and to guide placement of the eyes along the Y-axis. Referring to FIG. 5A, headrest 500 is also offset from optical axis 502 of a lens or set of lenses (not shown) by a fixed predetermined distance O along the Y-axis to further guide the position of the eyes along the Y-axis.

The compound-curved shape of headrest 500 is defined by a toroid, some example parameters of which are depicted in FIG. 5A. The toroid defining the shape of headrest 500 as shown in this example is a torus having a circular cross-section 504 with a radius R and a center 506 offset from optical axis 502 by a distance O′. Other revolved shapes that are not exact geometric circles may be used, as long as the shapes result in a user interface experience substantially similar to this example. As shown, the axis of revolution 508 of the torus intersects optical axis 502 at an angle beta (β). The radius from the axis of revolution 508 to the center 506 of the revolved cross-section 504 is the distance A. Focal point 510 along optical axis 502 is depicted as an example focal point for an example lens or lenses associated with a display unit in which headrest 500 is included. The distance between axis of revolution 508 and the line defining angle alpha (α) is the dimension C. The line 516 defining angle α relative to optical axis 502 intersects the point B, which is a nominal first point of contact between headrest 500 and a head of a user. This angle α can be selected to accommodate the combination of (1) natural variation of user forehead angle combined with (2) the range of head-to-viewer angles chosen by the user during ergonomic set-up.

In an example according to this disclosure, headrest optical axis offset O is approximately 33 millimeters, torus optical axis offset O′ is approximately 81 millimeters, torus radius R is approximately 70 millimeters, axis of revolution offset A is approximately 150 millimeters, axis of revolution angle β is approximately 70 degrees, and angle α is approximately 62 degrees. Other values for these dimensions may be used.

As noted, the cross-section in the Y-Z plane of headrest 500 (and other example headrests in accordance with this disclosure) defines a convex touch surface for a head of a user, which can have several advantages. First, the shape of example headrests can function to accommodate a wide range of user forehead shapes (i.e., in both sagittal/parasagital and transverse anatomical planes) and angles, as well as a wide range of user head-to-display unit set-ups. It can be uncomfortable for a user to press against a narrow edge of a cushion. The convex shape provided by the torus of example headrests in accordance with this disclosure can function to provide a comfortable rounded cushion for a range of user anatomies and display unit set-ups. This advantage may be provided whether the user is lightly touching the headrest/cushion, or the user is leaning in hard against the headrest/cushion (leading to the possibility of feeling the shape of the stiff frame beneath the soft cushion). Thus, users can lean in against the headrest with greater and lesser force throughout a procedure and still support a desirable eye position. Second, the torus shape has a reduced influence on the user ergonomic set-up. A flat or concave vertical surface tends to encourage the user to align the flat or concave vertical surface of the cushion with the surface their head, which may not be a good angle for visual or neck/torso comfort. Third, the shape of example headrests can accommodate a wide range of forehead/viewer angles with a thinner cushion than would be required for a flat or concave vertical surface cushion, because a flat or concave vertical surface cushion would need to compress to a relatively deeper depth to spread wide enough to feel aligned with the forehead. This relatively thinner cushion can improve user stability with reference to the lenses of a display unit to which the headrest is connected.

FIGS. 5B and 5C are perspective and exploded views depicting elements of contoured headrest 500. Referring to FIGS. 5B and 5C, headrest 500 includes frame 520, cover 522, cushion 524, and cover plate 526. Frame 520 includes cushion plate 528 and post 530, which is configured to be connected to a linear actuator of a display unit as described above. Frame 520 also includes fluid channel 532, which extends through cushion plate 528 and through at least a portion of post 530. Cover 522 includes shoulder 534, which extends around the periphery of the open end of cover 522 and which is sized and shaped to mate with shoulder 536 of cover plate 526. In an example, shoulder 534 is captured against cushion plate 528 with compression to provide a fluid-tight seal (e.g., a gas-tight seal) between the inside of cushion 524 and cover 522 to, e.g., improve sanitation.

Cover 522 envelops and surrounds the user-facing side of cushion 528, which is captured between cover 522 and cushion plate 528 of frame 520. Cover plate 526 is connected to cushion plate 528 by fasteners 538 (screws, bolts, latches, etcetera) and fixes and at least partially seals cover 522 around cushion 524. Shoulder 534 of cover 522 mates with and is captured by shoulder 536 of cover plate 526 and cushion plate 528 of frame 520.

A number of characteristics are important to the design of ergonomic headrests employed in telesurgical systems, including comfort (which can be characterized by the softness and resiliency of the headrest cushion), durability, and heat and moisture transfer away from the headrest user contact surface, as examples. Surgeons operating telesurgical systems may be operate a user control system to perform a procedure for extended periods of time. During the procedure, the surgeon can experience fatigue that may be intensified by poorly designed ergonomic elements of the user control system. Additionally, heat and moisture dissipation/transfer away from the headrest user contact surface can be important as the headrest against which the surgeon rests their head can become hot and moist.

In examples according to this disclosure, frame 520 can be made from a variety of materials. In some examples, frame 520 is made from a material that is configured to improve/provide heat transfer away from cover 524, cushion 526, and the head of the user using headrest 500 to improve user comfort. For example, frame 520 can be made from a material with a thermal conductivity that is sufficient to transfer heat away from cushion 526 and/or cover 524 to provide a comfortable experience for a user. In an example, frame 520 is made from a material that includes one or more of aluminum, magnesium, a carbon-filled polymer, and/or a ceramic.

To further facilitate heat transfer away from the user, frame 520 or some other portion of headrest 500 can include or incorporate a heat pipe, which is a heat-transfer device that employs phase transition to transfer heat between two solid interfaces. For example, at the hot interface of such a heat pipe, a volatile liquid in contact with a thermally conductive solid surface, e.g. a portion of cushion 524 and/or cushion plate 528 turns into a vapor by absorbing heat from that surface. The vapor then travels along the heat pipe to the cold interface and condenses back into a liquid, releasing the latent heat. The liquid then returns to the hot interface through capillary action, centrifugal force, gravity, as examples and the cycle repeats.

Additionally, frame 520 may optionally include fluid channel 532, which is configured to carry moisture and air into or away from cushion 526. For example, fluid channel 532 can function to carry moisture away from cushion 524 as well as the inside of cover 524 and cushion plate 528. Additionally, one or more fluid channels 532 may optionally be connected to a cooling system (not shown) that supplies air or another fluid coolant to cool, dehumidify, or both cool and dehumidify headrest 500. The cooling system could be any of a number of types of systems that are configured to modulate the temperature and/or the humidity of a heat load. In the example of FIGS. 5A-5C, fluid channel(s) 532 includes first channel 540 extending through post 530 into cushion plate 528 and a plurality of additional channels 540 extending through cushion plate and connected to the first channel.

In an example, channel(s) 530 functions to allow the softness of headrest 500 to be determined by the material properties of cover 522 and cushion 524, without gas trapped by cover 522 or cushion 524 acting as a balloon that reduces softness. In some examples, to aid in maintenance of an uncontaminated headrest 500 and the surrounding area, channel(s) 532 can be configured and positioned to terminate well within the housing or other outer boundary of the display unit to which headrest 500 to reduce the possibility of contaminants (e.g., sweat or cleaning fluids) from entering cushion 524 and then leaking back out into an area accessible to the user.

Headrest 500 and other headrests in accordance with this disclosure employ a cushion cover 522 that is separate from and envelops cushion 524. Separating cushion 524 and cover 522 into separate and distinct components is advantageous to headrest 500 because it allows the selection of different materials and associated properties for each component, which in combination may be more effective than a skinned elastomeric foam-based cushion. In an example, cover 522 is a non-porous, elastomeric cover, which functions to provide tactile comfort to users and also to guard against moisture ingress into cushion 524. In another example, cover 522 includes a silicone rubber, which provides durability, ease of cleaning, resistance to cleaning chemicals, biocompatibility, and cool conforming comfort to users. In another example, cover 522 includes a perfluoro elastomer.

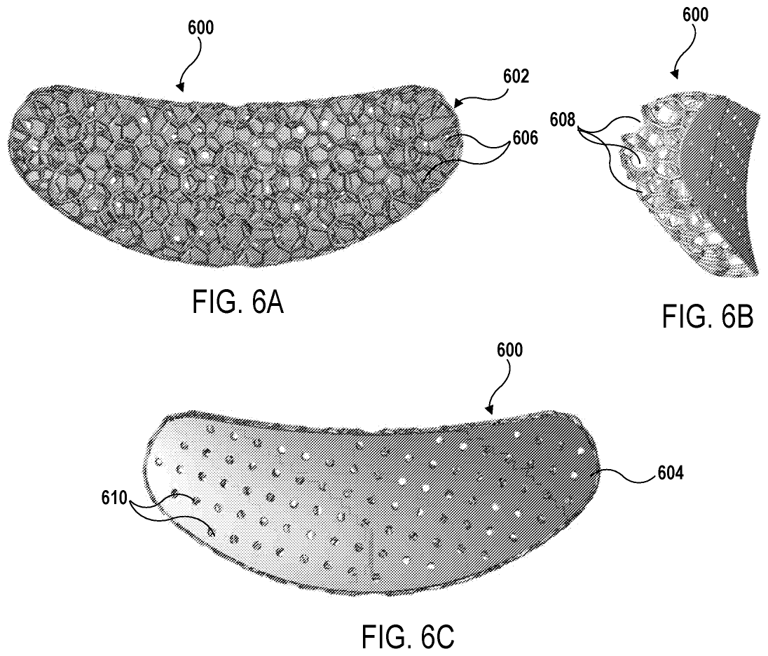

In examples of headrest 500 according to this disclosure, it is advantageous for cushion 524 (and other example headrest cushions) to provide appropriate levels of strength, durability, resilience, and breathability. In an example, cushion 524 is constructed from an elastomeric foam. In another example, however, cushion 524 is constructed from an elastomeric, three-dimensional lattice. An example of such a lattice cushion is depicted in FIGS. 6A-6C.

Referring to FIGS. 6A-6C, cushion 600 (which is optionally employed as cushion 524 in example headrest 500 of FIGS. 5A-5C) includes elastomeric, three-dimensional lattice 602 connected to backing plate 604. Lattice 602 includes a plurality of linear or curved elongated segments 606, each of which includes a first end and a second end between which each segment 606 extends. Each of the first end and the second end of each of segments 606 is connected to one or more ends of other segments or to backing plate 604 to define a lattice of segments bounding a plurality of material voids 608. Backing plate 604 includes a plurality of apertures 610, which can reduce the cost and weight and also provide ventilation into and out of cushion 600. Backing plate 604 functions to provide a relatively stiff backing for lattice 602, which can keep the lattice appropriately aligned (e.g., centered) in a headrest. In another example according to this disclosure, backing plate 604 includes attachment or alignment features to interface with cushion plate 528 or cover plate 526 of headrest 500. Additionally, in examples according to this disclosure, a cushion includes a three-dimensional lattice without a backing plate.

Employing a three-dimensional lattice 602 in cushion 600 may provide a number of advantages and/or benefits over other materials, e.g. elastomeric foams. In many cases, elastomeric foams provide poor heat transfer and create a hot environment for the users head. By contrast, an open three-dimensional lattice structure allows for convective heat transfer between the cushion cover and the headrest frame, thus providing a cooler touch surface. If this is combined with the aforementioned pressurized fluid flow, the advantage may be even greater. Additionally, by employing a three-dimensional lattice 602 in cushion 600, the shape and stiffness of the headrest may be engineered for improved balance between support for the eye position and user comfort. Moreover, three-dimensional lattice 602 can have variable stiffness across the cushion to further improve comfort.

Persons of skill in the art will understand that any of the features described above may be combined with any of the other example features, as long as the features are not mutually exclusive. All possible combinations of features are contemplated, depending on clinical or other design requirements. In addition, if manipulator system units are combined into a single system (e.g., telesurgery system), each individual unit may have the same configuration of features, or, one patient-side unit may have one configuration of features and another patient-side unit may have a second, different configuration of features.

The examples (e.g., methods, systems, or devices) described herein may be applicable to surgical procedures, non-surgical medical procedures, diagnostic procedures, cosmetic procedures, and non-medical procedures or applications. The examples may also be applicable for training, or for obtaining information, such as imaging procedures. The examples may be applicable to handling of tissue that has been removed from human or animal anatomies and will not be returned to a human or animal, or for use with human or animal cadavers. The examples may be used for industrial applications, general robotic uses, manipulation of non-tissue work pieces, as part of an artificial intelligence system, or in a transportation system.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. But, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description. Coordinate systems or reference frames are provided for aiding explanation, and implantations may use other reference frames or coordinate systems other than those described herein.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1.-35. (canceled)

36. A display unit of a user control system of a telesurgical system, the display unit comprising:
a display support structure;
a lens held within the display support structure and including an optical axis,
a headrest movably connected to the display support structure, the headrest being movable relative to the lens along a position adjustment axis parallel and offset with respect to the optical axis; and
an actuator that adjusts an offset distance between the headrest and the lens between at least a first position and a second position along the position adjustment axis, wherein the offset distance is greater in the second position than in the first position
wherein the headrest includes a curved user-facing surface that is angled, from an upper to a lower portion of the curved user-facing surface toward the support structure.

37. The display unit of claim 36, wherein:
the headrest includes a toroidal section defining a concave user-facing portion.

38. The display unit of claim 37, wherein:
the concave user-facing portion includes the curved user-facing surface; and
the curved user-facing surface is convex.

39. The display unit of claim 36, wherein:
the actuator comprises a linear actuator connected to and configured to move the headrest along the position adjustment axis.

40. The display unit of claim 39, wherein:
the display unit further comprises a user input control configured to control the linear actuator to translate the headrest along the position adjustment axis.

41. The display unit of claim 39, wherein:
the linear actuator is configured to move the headrest along the position adjustment axis in a range from a predetermined minimum extension distance and a predetermined maximum extension distance; and
the predetermined minimum extension distance and the predetermined maximum extension distance are based at least in part on a focal zone of the lens.

42. The display unit of claim 39, wherein:
the display unit further comprises a load sensor and a controller;
the load sensor is operatively connected to the linear actuator and communicatively connected to the controller; and
the controller is configured to:
receive one or more signals from the load sensor indicative of a load on the headrest; and
control the linear actuator to move the headrest with a force that is approximately equal and opposite in direction to the load on the headrest.

43. The display unit of claim 36, wherein:
the position adjustment axis is offset from the optical axis in a direction perpendicular to the optical axis by a first predetermined distance; and
the first predetermined distance is based at least in part on a focal zone of the lens.

44. The display unit of claim 36, wherein:
the lens is a first lens; and
the display unit further comprises a second lens below the headrest and next to the first lens.

45. A method of positioning a user relative to a display unit of a user control system of a telesurgical system, the method comprising:
presenting the display unit to the user,
the display unit including a movable headrest and a lens below the headrest,
the lens having an optical axis and a focal zone,
the headrest including a toroidal section defining a concave user-facing portion, and
the concave user-facing portion including a convex user-facing surface angled away from the user from an upper portion of the convex user-facing surface to a lower portion of the convex user-facing surface; and
on a condition in which the user is in contact with the headrest, moving the headrest to a position along a position adjustment axis that is parallel to the optical axis to place an eye of the user in the focal zone of the lens.

46. A display unit of a user control system of a telesurgical system, the display unit comprising:
lenses; and
a headrest moveable relative to the lenses, the headrest including:
a cushion plate,
a cover, and
a cushion captured between the cushion plate and the cover,
wherein the cushion includes a three-dimensional lattice.

47. The display unit of claim 46, wherein:
the three-dimensional lattice is connected to a backing plate that mates with the cushion plate.

48. The display unit of claim 47, wherein:
the backing plate of the cushion includes a plurality of apertures.

49. The display unit of claim 46, wherein:
the open three-dimensional lattice includes a plurality of linear or curved elongated segments;
each segment of the plurality of segments includes a first end and a second end; and
each of the first end and the second end of each of the plurality of segments is connected to one or more ends of other segments of the plurality of segments or to the backing plate to define a lattice of segments bounding a plurality of material voids.

50. The display unit of claim 46, wherein:
the cushion includes an elastomer.

51. The display unit of claim 46, wherein:
the cushion plate is in fluidic communication with the three-dimensional lattice.

52. The display unit of claim 51, further comprising a fluid delivery channel in fluidic communication with the cushion plate and the three-dimensional lattice.

53. A display unit of a user control system of a telesurgical system, the display unit comprising:
a headrest including:
a frame,
a cover, and
a cushion;
wherein the frame includes:
a cushion plate,
a post extending from the cushion plate, and
a fluid channel that extends through the cushion plate and through at least a portion of the post; and
wherein the cushion is captured between the cushion plate and the cover.

54. The display unit of claim 53, wherein:
the fluid channel includes a first channel and a plurality of second channels;
the first channel extends through the portion of the post and into the cushion plate; and
the plurality of second channels are each connected to the first channel and extend through the cushion plate.

55. The display unit of claim 53, wherein:
the display unit further includes a cooling system;
the cooling system is fluidically connected to the fluid channel; and
the cooling system is configured to supply a cooled fluid through the fluid channel to the cushion.

56. A display unit of a user control system of a telesurgical system, the display unit comprising:
a headrest including a frame and a cushion coupled to the frame;
wherein the frame includes a fluid inflow channel and a fluid outflow channel; and
wherein the fluid inflow channel and the fluid outflow channel are arranged so that a fluid flows through the fluid inflow channel into the cushion to transfer of heat away from the headrest.

57. The display unit of claim 56, wherein:
the display unit includes a cooling system;
the cooling system is fluidically connected to the fluid inflow channel; and
the cooling system is configured to supply the fluid through the fluid inflow channel to the cushion.

58. The display unit of claim 57, wherein:
the cooling system is fluidically connected to the fluid outflow channel; and
the cooling system is configured to receive the fluid from the cushion through the fluid outflow channel.

59. The display unit of claim 57, wherein:
the cooling system is configured to cool the fluid before the fluid enters the fluid inflow channel.
